(19) United States
(12) Patent Application Publication
(10) Pub. No.: US 2005/0244360 A1
(43) Pub. Date: Nov. 3, 2005

(54) Cosmetic composition for greasy skin care, containing a carboxylic fatty acid or a derivative thereof

(75) Inventor: Nelly Billoni, Valmondois (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: L'OREAL, PARIS (FR)

(21) Appl. No.: 11/176,350

(22) Filed: Jul. 8, 2005

(30) Foreign Application Priority Data
Aug. 1, 2003 (FR) 0300156
Jan. 8, 2004 (WO) PCT/FR04/00028

(51) Int. Cl. A61K 7/075; A61K 7/08; A61K 7/06

(52) U.S. Cl. 424/70.22; 424/74

(57) ABSTRACT

The invention concerns a cosmetic composition for treating or preventing disorders associated with a greasy skin, in particular by an activity reducing sebum secretion, comprising as active principle at least one dicarboxylic fatty acid or one derivative of such a fatty acid. The invention concerns in particular cosmetic compositions for topical administration. The invention also concerns a cosmetic method for treating or preventing disorders associated with hyperseborrhea.

### CROSS-REFERENCE TO PRIORITY/PCT/PROVISIONAL APPLICATIONS

This application claims priority under 35 U.S.C. § 119 of FR 03/00156, filed Jan. 8, 2003 and of provisional application Ser. No. 60/460,414, filed Apr. 7, 2003, and is a continuation of PCT/FR 2004/000028, filed Jan. 8, 2004 and designating the United States (published in the French language on Aug. 19, 2004 as WO 2004/069219 A1; the title and abstract were also published in English), each hereby expressly incorporated by reference and each assigned to the assignee hereof.

### BACKGROUND OF THE INVENTION

1. Technical Field of the Invention

The present invention relates to a cosmetic composition intended to correct or prevent disorders associated with oily skin, in particular by a sebum secretion reduction action, comprising as the active principle at least one dicarboxylic fatty acid or a derivative of said fatty acid. In particular, the invention provides cosmetic compositions intended for topical administration. The invention also pertains to a cosmetic method for correcting or preventing disorders associated with hyperseborrhea.

2. Description of Background and/or Related and/or Prior Art

The terms “oily skin” and “dry skin” are normally used as opposites. However, they are not actually opposites, and this leads to confusion. Two parameters should be considered when classifying skin: (1) the degree of lipid load, i.e., whether the skin appears oily or not, and (2) the degree of hydration. The simplest skin type classification is based on these two parameters. Depending on the degree of hydration of the corneous layer and its sebum production, then, three skin types can be distinguished:

dry skin;

oily skin;

combination skin.

Thus, oily skin is hyperseborrheic and characterized by exaggerated secretion and excretion of sebum. Conventionally, a sebum rate of more than 200 μg/cm² on the forehead is considered to be characteristic of oily skin. Oily skin is skin with a shiny appearance, thick and with follicular orifices which are dilated or blocked with tiny corneous spicules or comedones (which, however, result from retention rather than from an increase in excretion). Oily skin is frequently associated with desquamation problems and with a thick skin grain. A further disadvantage of oily skin is that makeup does not stay on at all well, which means that the skin must be treated before applying makeup to limit the flow of sebum.

In addition to the above aesthetic problems, excess sebum may act as a support in the anarchic development of the bacterial flora saprophyte (in particular Propionibacterium acnes and Pityrosporum ovale), and causes comedones and/or acne lesions.

However, acne and oily skin do not always go hand in hand. Indeed, while acne is frequent in cases of androgenic hyperseborrhea (typically in the case of juvenile acne), it may develop in individuals whose sebum production is not particularly high. Conversely, individuals with oily skin do not systematically develop acne lesions.

Sebum is a natural product from the sebaceous gland which constitutes a part of the pilosebaceous unit. It is essentially a simple or complex mixture of lipids. Conventionally, the sebaceous gland produces squalene, triglycerides, aliphatic waxes, cholesterol waxes and, possibly, free cholesterol (Stewart, 1992). The action of bacterial lipases converts a variable part of the triglycerides formed into free fatty acids.

The sebocyte constitutes the competent cell of the sebaceous gland. Sebum production is associated with a terminal differentiation programme in this cell. During said differentiation, the metabolic activity of the sebocyte is essentially orientated towards lipid biosynthesis (lipogenesis), and more precisely towards fatty acid neosynthesis.

Thus, there exists a need for a cosmetic composition which can prevent the aesthetic problems and the development of acne lesions associated with sebum overproduction. A number of cosmetic compositions which can offer a solution to said problems have been proposed in the prior art. Such compositions primarily comprise, as the active principle:

powders which can absorb sebum and thus mattify the skin by a mechanical effect;

alpha or beta-hydroxyacids which, by their keratolytic effect, encourage expulsion of corneous plugs;

astringent agents which can control sebaceous follicle dilation;

exfoliant agents which can reduce the thickness of the skin; and

active agents which act on biochemical mechanisms, in particular the enzymes involved in sebum production.

However, the efficacy of such compositions is relative, as the majority do not limit sebaceous production but only absorb the sebum.

The present invention aims to provide means for treating the fundamental problem with oily skin by controlling lipogenesis rather than treating the consequence of excessive lipogenesis ex post facto.

During studies of compounds that may have sebo-regulatory properties, the inventors have demonstrated that an aliphatic C9 dicarboxylic acid, the azelaic acid, can significantly inhibit lipogenesis.

This compound has already been used in a pharmaceutical preparation intended for acne treatment, namely SKINOREN® (SHERING laboratories).

Acne is a disease of the pilosebaceous unit, essentially characterized by inflammation. It particularly affects the facial and upper dorsal zones and appears most frequently in adolescence (Shalita, 1998). Acne generally involves (1) increased production of sebum (with respect to normal conditions); (2) hyperkeratinization of the infra-infundibulum (comedone); (3) hyperproliferation of commensal flora; and (4) inflammation.

Many methods have been proposed to treat this disorder, from changing the diet to using drugs which act on the endocrine system (progesterone and oestrogen in particular).

In particular, the use of compounds with an antibacterial action for topical application has been proposed. In this respect, Bladon et al. compared the efficacy of two compounds with an antibacterial action, tetracycline and azelaic acid, during a study for the treatment of acne vulgaris, carried out as a double blind study on 45 male subjects with clinical acne. They demonstrated that topical application of a gel containing 20% azelaic acid could reduce the number of cutaneous micrococcaceae and Propionibacterium sp by a factor of 224 and 30 respectively (Bladon, 1986). In the same study, the authors measured the effect of azelaic acid on sebum excretion using a separate group of 11 individuals with physiological acne, and showed that topical application of a 20% azelaic acid gel did not cause a significant modification in sebum excretion.

In a subsequent study, Mayer-da-Silva et al showed that, at the doses used to treat acne, in particular in SKINOREN®, azelaic acid did not modify either the quantity or the composition of the excreted sebum, neither in subjects with acne nor in subjects with seborrheic skin who did not suffer from acne, nor in normoseborrheic subjects (Mayer-da-Silva, Gollnick et al., 1989).

EP-A-0,705,602 describes the use of α-ω alkanedioic acids as active agents against cutaneous surinfections. Azelaic acid is cited as an agent for use in this context.

In WO-A-98/20834, Tamarkin notes the inability of azelaic acid to reduce sebum production and describes a keratolytic effect for that acid, which reduces the proliferation of human keratinocytes when used at concentrations of the order of 1 mM.

More generally, it is of particular interest to note that while certain of the antibacterial treatments mentioned above have proved effective in reducing the number and intensity of acne lesions, very few have recorded a reduction in the sebum hypersecretion associated with acne (Thiboutot, 2000).

For this reason, it is not considered trivial to imagine an application to the treatment of hyperseborrheic oily skin which is non-acneic or pre-acneic for an active molecule which is used effectively in treating acne.

### SUMMARY OF THE INVENTION

As will be shown in the experimental section below, Applicant has, surprisingly and paradoxically when considering the prior art, shown that azelaic acid can significantly inhibit lipogenesis. In fact, application of 1 μM—i.e., about 0.02×10⁻¹%—of azelaic acid to a model immortalized culture of human sebocytes caused a 7% reduction in lipid secretion (Example 1). This concentration, which is much lower than those used in dermatological applications for this compound (1000 times lower than that used by Tamarkin in WO-A-98/20834, to observe the onset of a keratolytic effect), can produce results which are very different from those obtained at higher concentrations, wherein the application of azelaic acid causes no modification in the quantity nor the composition of the excreted sebum.

Thus, in a first aspect, the present invention concerns a cosmetic composition for the care of oily skin, containing at least one fatty dicarboxylic acid or one of its active derivatives.

In the text below, the concentrations of fatty dicarboxylic acid are expressed in molarities. To facilitate conversion of these concentrations into percentages, note that for azelaic acid in a standard aqueous solution, a concentration of 1M corresponds to 20% azelaic acid. Clearly, the skilled person will be capable of adjusting this correspondence as a function of the fatty dicarboxylic acid used and of the other elements of the composition.

The cosmetic compositions of the invention advantageously include a concentration in the range 1 nM to 1 mM of active principle for the care of oily skin, preferably less than 0.5 mM, for example in the range 1 nM to 0.1 mM, and more preferably in the range 0.01 μM to 0.1 mM, or even in the range 0.01 μM to 10 μM. The activity of the cosmetic composition of the invention is preferably manifested by a reduction in the amount of sebum in the treated skin.

In general in the context of the invention, it is considered that compounds are active in oily skin lipogenesis when their use for the care of oily skin results in a reduction in the amount of sebum in the skin of at least 10%, preferably at least 30%, the amount of sebum being measured using a type SM810 sebumeter sold by Courage & Khazaka (Cologne, Germany).

### DETAILED DESCRIPTION OF BEST MODE AND SPECIFIC/PREFERRED EMBODIMENTS OF THE INVENTION

In the compositions of the invention, the fatty dicarboxylic acid derivative is preferably selected from sugar esters, amides or fatty alcohol esters, in particular non keratolytic fatty alcohol esters. In particular, the fatty alcohols that can be used in the context of the present invention will preferably be distinct from the following acids: ortho-, meta- and para-hydroxybenzoic acids, ortho-, meta- and para-hydroxyalkyl benzoates, ortho-, meta- and para-dihydroxybenzene, ortho-, meta- and para-hydroxytoluene, and derivatives thereof. Preferred amines that can be used are ammonia or primary or secondary amines, for example amino acids or aminosugars. Any type of sugar can be used in the context of the present invention. Monosaccharides or disaccharides will, however, be used in preference, as well as pyrannic hexoses. Non-limiting examples of preferred sugars for use in the present invention that can be cited are lactose, sorbitol, mannitol, erythritol, ketoses and aldoses such as arabinose or glucose. Preferably, the fatty dicarboxylic acid or the fatty dicarboxylic acid portion of the derivative present in the cosmetic composition is linear and contains 8, 9 or 10 carbon atoms.

A cosmetic composition of the invention may also comprise a plurality of different fatty dicarboxylic acid derivatives. In said composition, the fatty dicarboxylic acid portion of each of said derivatives is preferably linear and contains 8, 9 or 10 carbon atoms.

A particular cosmetic composition of the invention contains azelaic acid, for example in a concentration in the range 0.1 μM to 10 μM.

In a cosmetic composition of the invention which comprises a plurality of compounds selected from fatty dicarboxylic acids and their derivatives, each of said components is a pure molecule, a plant extract or a microorganism extract. Clearly, in such a composition, the compounds may, independently of each other, be present in the pure form or in the form of a plant or microorganism extract.

One particular implementation of the invention is a cosmetic composition as described above, which contains an extract of Malassezia furfur and/or of Pityrosporum ovale and/or of Pityrosporum orbiculare.

The extract in question can be obtained using any method which is known to the skilled person. After culturing microorganisms, the skilled person can separate the microorganisms and the culture medium, for example by filtration, coagulation and/or freeze drying or, as is preferable, by centrifugation. The culture medium and the microorganisms may be autoclaved and frozen. The microorganisms may also be freeze dried. The extract may be obtained either from microorganisms or from the culture medium, using any technique that is known to the skilled person.

In a variation of the invention, a fatty dicarboxylic acid constituting the cosmetic active principle may be associated with different active principles which are also selected for their beneficial action on oily skin, and in particular for their action in reducing lipogenesis.

The cosmetic compositions of the invention are preferably topically applicable, in particular to non acneic or preacneic hyperseborrheic oily skin or to an oily scalp.

The invention also pertains to a composition as described above, characterized in that it contains various cosmetically active principles with a lipogenesis reduction activity.

A composition formulated for topical administration of the invention contains a cosmetically or dermatologically acceptable medium, i.e., a medium that is compatible with the skin, nails, mucosae, tissues and hair. In a preferred implementation of the invention, the pH of the composition is preferably close to that of the skin, in the range 4 to 7. The composition comprising a fatty dicarboxylic acid derivative or a derivative thereof is preferably applied topically, to the face, neck, hair, mucosae and nails or to any other cutaneous region of the body.

A cosmetic composition of the invention can be in the form of any of the galenical forms that are normally used in the cosmetic and dermatological fields. In particular, it can be in the form of hydroalcoholic or oily solutions, lotion or serum type dispersions, anhydrous or oily gels, emulsions with a liquid or semi-liquid milk type consistency, obtained by dispersing an oily phase in an aqueous phase (O/W) or vice versa (W/O), or a triple emulsion (W/O/W or O/W/O), suspensions or emulsions of a soft, semi-solid or solid cream type consistency, gel, microemulsions, or micro-capsules, micro-particles, or ionic and/or non ionic type vesicular dispersions. These compositions are prepared using the usual methods.

A preferred and especially suitable form for the compositions of the invention for the treatment of disorders associated with hyperseborrhea is an oil-in-water type emulsion. More preferably, the oily phase of said emulsion has a composition that is close to that of sebum, i.e., its components are the principal constituents of sebum (squalene, triglycerides, aliphatic waxes, cholesterol waxes and, possibly, free cholesterol), or constituents with an equivalent structure, present in proportions similar to those present in sebum.

As an indication, an oily phase mimicking the composition of sebum which can be used in the context of the present invention is composed of at least 12% to 18% of squalene, 0 to 10% of trimyristin, 0 to 10% of trimyristolein, 0 to 10% of tripalmitin, 0 to 10% of tripalmitolein, 0 to 20% of triolein, 0 to 10% of tristearin, 0 to 60% of glycerol, 0 to 15% of oleic acid, 0 to 10% of palmitic acid, 0 to 15% of palmitoleic acid, 0 to 10% of myristic acid, 0 to 10% of myristoleic acid, 5% to 20% of myristyl oleate, 5% to 10% of palmityl oleate, 0.5% to 3% of cholesteryl palmitate and 0 to 5% of cholesterol.

Other oily phases that mimic the composition of sebum which can be used in the context of the present invention are described in FR-A-0,114,033, GB-889688, WO-A-02/26207 and WO-A-02/11693, from which a formulation can be cited which comprises 35% to 70% of macadamia oil, 18% to 30% of jojoba oil (wax), 5% to 15% of avocado oil, 6% to 15% of a non-saponifiable olive oil product, 0.5% to 3% of free cholesterol, 0.5% to 1.5% of cholesterol oleate,1% to 3% of soya phospholipids, and 0.05% to 0.15% of butylhydroxytoluene.

The advantage of a composition the oily phase of which mimics the composition of sebum is to allow better availability of the active principle in the target organ, i.e., the sebaceous gland.

The compositions of the invention can also be used for the hair in the form of alcoholic or hydroalcoholic solutions or in the form of creams, gels, emulsions or foams. They can be used by application to the scalp or, if appropriate, can constitute treatment shampoos for oily hair.

The quantities of the various constituents of the compositions used in the invention are those which are conventionally used in the fields under consideration.

Said compositions constitute protective, treatment or care creams for the face, for the hands or for the body, body milks for care or protection, lotions, gels or foams for care of the skin or mucosae or for cleaning the skin. They are of particular advantage in treating the oiliest zones of the body, in particular the face, neck and back.

The compositions can also consist of solid preparations constituting soaps or cleansing bars.

In known manner, the composition of the invention may also contain adjuvants that are normal in the cosmetic and dermatological fields, such as hydrophilic or lipophilic gelling agents, hydrophilic or lipophilic active agents, preservatives, antioxidants, solvents, fragrances, fillers and colorants. The quantities of these various adjuvants are those which are conventionally used in the fields under consideration, for example 0.01% to 20% of the total composition weight. Clearly, the skilled person will take care to select any additive or additives and/or their quantities such that the advantageous intrinsic properties of the composition are in accordance with the invention, in particular so that the sebo-regulatory activity of the fatty dicarboxylic acid or its derivative is not, or is not substantially, altered by the envisaged additions.

Oils which can be used in the invention which can be cited are mineral oils (Vaseline oil), vegetable oils (shea oil, sweet almond oil), animal oils, synthesized oils, silicone oils (cyclomethicone) and fluorinated oils (perfluoropolyethers). It is also possible to use fatty alcohols, fatty acids (stearic acid), waxes (paraffin, carnauba, beeswax) as the fatty material.

Emulsifying agents which can be used in the context of the invention that can be cited are polysorbate 60 and sorbitan stearate sold by ICI as Tween 60 and Span 60 respectively. Co-emulsifying agents can be added, such as PPG-3 myristyl ether sold by Witco as Emcol 249-3K.

Solvents which can be used in the invention that can be cited are lower alcohols, in particular ethanol, isopropanol and propylene glycol.

Hydrophilic gelling agents which can be cited are carboxyvinyl polymers (carbomers), acrylic copolymers such as acrylate/alkylacrylate copolymers, polyacrylamides, polysaccharides such as hydroxypropylcellulose, natural gums (xanthan) and clays, and lipophilic gelling agents which can be cited are modified clays such as bentonites, metallic salts of fatty acids such as aluminum stearates, hydrophobic silica, polyethylenes and ethylcellulose.

Hydrophilic active agents which can be used are proteins or protein hydrolysates, amino acids, polyols, urea, allantoin, sugars and sugar derivatives, hydrosoluble vitamins, starch, and bacterial or plant extracts, in particular aloe vera.

Lipophilic active agents which can be used are tocopherol (vitamin E) and its derivatives, essential fatty acids, ceramides and essential oils.

In order to combat photoaging effectively, it is also possible to add to the composition of the invention one or more complementary sun screens which are active in the UV-A and/or UV-B region, which are hydrophilic or lyophilic, optionally comprising a sulfonic function.

The sun screen is preferably selected from organic filters and/or mineral filters.

Organic filters which can be cited are cinnamic derives, salicylic derivatives, camphor derivatives, triazine derivatives, benzophenone derivatives, dibenzoylmethane derivatives, β, β-diphenylacrylate derivatives, p-aminobenzoic acid derivatives, polymer filters and silicone filters described in WO-A-93/04665 or organic filters described in EP-A-0,487,404.

Mineral filters which can be cited are pigments or nanopigments (mean primary particle size: generally between 5 nm and 10 nm, preferably between 10 and 50 nm), metal oxides which may or may not be coated, such as nanopigments of oxides of titanium (amorphous or crystalline in the form of rutile and/or anatase), iron, zinc, zirconium or cerium, which are all photoprotective agents which are well known per se, acting by physically blocking (reflection and/or diffusion) the UV radiation. Conventional coating agents include alumina and/or aluminum stearate. Such nanopigments of metal oxides, whether coated or not, have been described in particular in EP-A-0,518,772 and EP-A-0,518,773.

Examples of complementary sun screens which are active in the UV-A and/or UV-B which can be cited are:
p-aminobenzoic acid;
oxyethylenated (25 mol) p-aminobenzoate;
2-ethylhexyl p-dimethylaminobenzoate;
N-oxypropylenated ethyl p-aminobenzoate;
glycerol p-aminobenzoate;
homomenthyl salicylate;
2-ethylhexyl salicylate;
triethanolamine salicylate;
4-isopropylbenzyl salicylate;
4-ter-butyl-4′-methoxy-dibenzoylmethane (PARSOL 1789 from GIVAUDAN ROURE);
2-ethylhexyl p-methoxycinnamate (PARSOL MCX from GIVAUDAN ROURE);
4-isopropyl-dibenzoylmethane (EUSOLEX 8020 from MERCK);
menthyl anthranilate;
2-ethylhexyl-2-cyano-3,3′-diphenylacrylate (UVINUL N359 from BASF);
ethyl-2-cyano-3,3′-diphenylacrylate;
2-phenyl benzimidazole-5-sulfonic acid and its salts;
3-(4′-trimethylammonium)-benzyliden-bornan-2-one-methylsulfate;
2-hydroxy-4-methoxybenzophenone (UVINUL MS 40 from BASF);
2-hydroxy-4-methoxybenzophenone-5-sulfonate (UVINUL MS 40 from BASF);
2,4-dihydoxybenzophenone (UVINUL 400 from BASF);
2,2′-4,4′-tetrahydroxybenzophenone (UVINUL D 50 from BASF);
2,2′-dihydroxy-4,4′-dimethoxybenzophenone (HELISORB II from NORQUAY);
2-hydroxy-4-n-octoxybenzophenone;
2-hydroxy-4-methoxy-4′-methylbenzophenone;
α-(2-oxoborn-3-ylidene)-tolyl-4-sulfonic acid and its salts;
3-(4′-sulfo)benzyliden-bornan-2-one and its salts;
3-(4′-methylbenzylidene)-d,l-camphor;
3-benzylidene-d,l-camphor;
benzene 1,4-di(3-methylidene-10-camphosulfonic) acid and its salts (MEXORYL SX from CHIMEX);
urocanic acid;
2,4,6-tris-[p-(2′-ethylhexyl-1′-oxycarbonyl)anilino]-1,3,5-triazine;
2-[p-(tertiobutylamido)aniline]-4,6-bis[p-(2′-ethylhexyl-1′-oxycarbonyl)anilino]-1,3,5-triazine;
2,4-bis{[4-2-ethylhexyloxyl]-2-hydroxylphenyl}-6-(4-methoxyphenyl)-1,3,5-triazine;
N-(2 and 4)-[2-oxoborn-3-ylidene)methyl)benzyl]-acrylamide polymer;
4,4-bis-benzimidazolyl-phenylene-3,3′,5,5′-tetrasulfonic acid and its salts;
2,2′-methylene-bis-[6-(2H-benzotriazol-2-yl)-4-(1,1,3,3-tetramethylbutyl)phenol];
polyorganosiloxanes with a malonate function.

The invention also pertains to a cosmetic treatment method, by applying compositions as defined above using the technique which is normal in using said compositions. An example is:

application of creams, gels, serums, pomades, lotions, milks to the skin, scalp, nails and/or mucosae.

A cosmetic method for treating oily skin for the prevention and/or treatment of acne lesions consisting of application to the skin, mucosae and/or keratinous fibers of a composition containing at least one fatty dicarboxylic acid or a derivative thereof also forms an integral part of the present invention.

A particular category of subjects who may benefit from a cosmetic treatment of the invention is the category of persons with non acneic hyperseborrheic skin. As mentioned above, sebum hypersecretion is not systematically associated with acne, but it is nonetheless not aesthetically pleasing and there is currently no effective treatment.

Against all expectations, the inventors have demonstrated in the experimental section below that concentrations of fatty dicarboxylic acid of the order of 1 μM (thus, about 0.00002%), i.e., much lower than those previously used in anti-acne applications, can produce a significant reduction in the production of sebum by sebocytes.

A particular cosmetic method of the invention is a method for treating oily skin for the prevention and/or treatment of acne lesions consisting of application to the skin, mucosae and/or keratinous fibers of a composition containing a fatty dicarboxylic acid (or one of its active derivatives) in a concentration which can supply the sebocytes with a quantity of dicarboxylic acid corresponding to the concentration of azelaic acid the efficacy of which has been observed in vitro. Clearly, the skilled person will select this concentration as a function of the derivative concerned and the excipients in the composition, to provide suitable vectoring of the active compound towards the sebocytes.

By way of indication, a composition used in a cosmetic method of the invention may comprise azelaic acid in a concentration in the range 0.1 μM to 10 μM.

More generally, in particular in the context of methods for regulating the secretion of sebum in subjects with non acneic hyperseborrheic skin, the compositions used will comprise a concentration of fatty dicarboxylic acid (or one of its active derivatives) of 500 mM or less, preferably 50 mM or less, more preferably 5 mM or less, or even 0.5 mM or less.

The invention also pertains to a cosmetic method for regulating hyperseborrhea in non acneic subjects, consisting of applying to the skin, mucosae and/or keratinous fibers a composition containing at least one fatty dicarboxylic acid or one of its derivatives present in said composition in a concentration in the range 1 nM to 50 nM, preferably 5 mM or less and more preferably 0.5 mM or less.

In particular, the invention pertains to the use of azelaic acid for the preparation of a sebo-regulating composition, for the treatment of oily skin. The invention also pertains to the use of azelaic acid for the preparation of a composition for correcting disorders associated with hyperseborrhea.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Reduction of Lipogenesis

The test which was developed consisted of measuring, after 2 days of treatment, the quantity of lipids produced by sebocytes from an immortalized human line (in the presence or absence of active agents). The fluorescence emitted by RED NILE dye was measured at two distinct wavelengths to quantify the amount of characteristic neutral lipids of the sebum.

TABLE 1

| | QUANTITY OF | REDUCTION IN |
|---|---|---|
| PRODUCT | LIPIDS MEASURED | LIPOGENESIS/CONTROL |
| Cortisol, 0.1 μM | 71 | −29% |
| Azelaic acid, 1 μM | 93 | −7% |
| Control | 100 | / |

Glucocorticoids are known to reduce the production of sebum in non acneic subjects (Leveque, J. L. et al., (1991)). Thus, cortisol was selected as a reference, while the culture medium alone was selected as the control (no supplemental excipient was added either to the cortisol or to the azelaic acid, these latter being dissolved in the culture medium under the test conditions). These experiments showed that azelaic acid inhibits sebum production in a concentration of 1 μM. Its activity was moderate compared with the pharmaceutical reference. Nevertheless, it should have a significant cosmetic action in a suitable formulation, if necessary by making repeated applications.

### EXAMPLE 2

### Tolerability

The tolerability of azelaic acid was measured by measuring the cytotoxicty of the product using SZ 95 sebocytes (Zouboulis, Seltmann et al., 1999). The test conditions were the same as those given in Example 1. The cytotoxicity was measured by the production of LDH in the culture medium.

TABLE 2

| PRODUCT | LDH VARIATION/CONTROL |
|---|---|
| Azelaic acid, 1 μM | −20% |

As can be seen from Table 2 above, azelaic acid improves sebocyte survival under the experimental conditions. Its seboregulatory activity could not therefore be attributed to cytotoxicity.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

### REFERENCES

Bladon, P T, B M Burke et al (1986). “Topical azelaic acid and the treatment of acne: a clinical and laboratory comparison with oral tetracycline”, Br J Dermatol 114(4): 493-9.

Leveque J L, C Pierard-Franchimont et al, (1991). “Effect of topical corticosteroids on human sebum production assessed by two different methods” Arch Dermatol Res 283(6): 372-6.

Mayer-da-Silva A, H Gollnick et al (1989). “Effects of azelaic acid on sebaceous gland, sebum excretion rate and keratinization pattern in human skin. An in vivo and in vitro study”. Acta Derm Venereol Suppl. 143: 20-30.

Shalita, A R (1998). “Clinical aspects of acne” Dermatology 196(1): 93-4.

Stewart M E (1992). “Sebaceous gland lipids”. Semin Dermatol 11(2): 100-5.

Thiboutot D (2000). “New treatments and therapeutic strategies for acne” Arch Fam Med 9(2): 179-87.

Zouboulis C C, H Seltmann et al (1999). “Establishment and characterization of an immortalized human sebaceous gland cell line (SZ95)”. J Invest Dermatol 113(6): 1011-20.

1. A cosmetic composition for the care of oily skin, characterized in that it contains at least one fatty dicarboxylic acid or one of its active derivatives, in a concentration of less than 0.5 mM, preferably in the range 1 nM to 0.1 mM, more preferably in the range 0.01 μM to 10 μM.

2. A cosmetic composition according to claim 1, in which the fatty dicarboxylic acid is selected from fatty alcohol esters, sugar esters and amides.

3. A cosmetic composition according to claim 1, characterized in that the fatty dicarboxylic acid or the fatty dicarboxylic acid portion of the derivative is linear and contains 8, 9 or 10 carbon atoms.

4. A cosmetic composition according to claim 1, comprising a plurality of fatty dicarboxylic acid derivatives in which the fatty dicarboxylic acid portion is linear and contains 8, 9 or 10 carbon atoms.

5. A cosmetic composition according to claim 1, containing azelaic acid in a concentration in the range 0.1 μM to 10 μM.

6. A cosmetic composition according to claim 1, in which each fatty dicarboxylic acid or fatty dicarboxylic acid derivative is a pure molecule, a plant extract or a microorganism extract.

7. A cosmetic composition according to claim 6, containing an extract of Malassezia furfur and/or Pityrosporum ovale and/or Pityosporum orbiculare.

8. A cosmetic composition according to claim 1, characterized in that it is applicable topically.

9. A cosmetic composition according to claim 1, for the care of hyperseborrheic, non acneic or pre-acneic oily skin, or for the oily scalp.

10. A cosmetic composition according to claim 1, characterized in that it contains different cosmetically active principles having lipogenesis reduction activity.

11. A cosmetic composition according to claim 1, characterized in that it is constituted by an oil-in-water emulsion, in which the composition of the oily phase is close to that of sebum.

12. A cosmetic method for the treatment of oily skin and/or for the prevention of acne lesions comprising application to the skin, mucosae and/or keratinous fibers of a composition containing at least one fatty dicarboxylic acid or one of its derivatives.

13. A cosmetic method for the treatment of oily skin and/or for the prevention of acne lesions, comprising application to the skin, mucosae and/or keratinous fibers of a composition containing azelaic acid in a concentration in the range 0.1 μM to 10 μM.

14. Use of azelaic acid for the preparation of a sebo-regulating cosmetic composition, for the care of oily skin.

15. Use according to claim 14, for the preparation of a composition intended to correct disorders associated with hyperseborrhea.
